(19) United States
(12) Patent Application Publication
(10) Pub. No.: US 2006/0078598 A1
(43) Pub. Date: Apr. 13, 2006

(54) Animal feed containing an encapsulated ingredient

(75) Inventors: Patrick Andrew Jobe, Becker, MN (US); Bruce Brim McGoogan, Plymouth, MN (US); Pierre P. Frumholtz, Itrapuato (MX)

Correspondence Address: CARGILL, INCORPORATED, LAW/24, 15407 MCGINTY ROAD WEST, WAYZATA, MN, 55391, US

(73) Assignee: CAN Technologies, Inc.

(21) Appl. No.: 11/282,544

(22) Filed: Nov. 18, 2005

Related U.S. Application Data
(62) Division of application No. 10/338,515, filed on Jan. 8, 2003.
(60) Provisional application No. 60/346,668, filed on Jan. 8, 2002.
(60) Provisional application No. 60/350,581, filed on Jan. 22, 2002.

(51) Int. Cl. A23K 1/165; A23L 1/18; A61K 47/00

(52) U.S. Cl. 424/442; 426/625

(57) ABSTRACT

An encapsulated ingredient is disclosed comprising an ingredient component and a coating component. The coating component includes a lipid and one or more hydrophobic, high melting point compounds. The coating component typically has a melting point of at least about 70° C.

### CROSS-REFERENCE TO RELATED PATENT APPLICATIONS

This application is a divisional of U.S. patent application Ser. No. 10/338,515, entitled “Encapsulation by Coating with a Mixture of Lipids, and Hydrophobic, High Melting Point Compounds,” filed on Jan. 8, 2003, which claims priority of U.S. Provisional Application Ser. No. 60/350,581, filed on Jan. 22, 2002, and U.S. Provisional Application Ser. No. 60/346,668, filed on Jan. 8, 2002, all of which are incorporated herein by reference in their entireties.

### BACKGROUND

In preparing animal feed, it is important to ensure that the feed has the proper physical characteristics and provides the proper nutrition for the animals. Traditional animal feed such as naturally occurring vegetation may not provide essential ingredients or nutrients necessary for the optimum health and performance of the animal. To overcome this problem, animal feed may be supplemented with a large variety of ingredients targeted to provide the animal with the proper nutrition. Further it may be desirable to modify the physical characteristics of the feed or portions of the animal feed.

Unfortunately, during the feed manufacturing process (generally extrusion, pelleting, etc.), high temperature and pressure conditions often result in a significant loss of valuable heat sensitive and/or water-soluble ingredients. Ingredients may also be lost in post-manufacturing processes when feeds are exposed to air or water during, for example, storage and handling, or from conditions that occur in the animal's own system. The loss of ingredient value or functionality can be costly and increase the risk of missing a targeted feed composition that is necessary for optimal performance of the animal.

There have been previous attempts to protect some of the more susceptible or costly ingredients by coating them with various materials. Approaches practiced to date include coating combinations of chemically altered ingredients with various materials to achieve protection. A common, inexpensive technique used, was to coat the ingredient with some form of lipid using a process such as micro encapsulation, but satisfactory results were generally limited to processing environments below 70° C.

Many of the lipids used to coat ingredients for protection have been fats or fatty acids derived from a variety of animal and vegetable sources. Waxes, tallows, lower chain saturated and monounsaturated fatty acids and tristearins are some examples of the lipids that are currently used for lipid only coatings. Such coatings all have a common characteristic in that their melting points commonly do not exceed about 70° C. Because the melting points of these lipids are typically no higher than 70° C., their effective use as a protective coating is usually limited to processes having a temperature range below 70° C. The temperatures associated with extrusion and pelleting processes are typically greater than 70° C. and feeds produced by extrusion often require drying at temperatures exceeding 100° C., thus rendering lipid only coatings largely ineffective for maximum ingredient protection when using such high heat manufacturing processes.

Many past coatings, lipid or otherwise, were not effective for a number of further reasons. Many coatings effectively protect the ingredients from temperature and pressure effects but were too costly to produce. Other inexpensive coatings did not effectively protect ingredients from high temperature and pressure conditions encountered in the manufacturing process. Many of the coatings failed to effectively stabilize the ingredients under common storage and use conditions. Still other prior coatings failed to provide the necessary bioavailability in the intended end user, typically a ruminant or aquaculture species. In addition to these problems, some past coatings had the additional disadvantage of using volatile solvents as part of the process of making the coating and using the coating to coat the ingredient.

### SUMMARY

The present compositions relate to the protection of ingredients through prevention of physical loss and/or loss of functionality which can arise due to conditions associated with manufacturing, storage and/or use. Examples of conditions which can lead to such losses include manufacturing operations such as pelleting or extrusion, as well as post manufacturing leaching or biological losses. One particularly valuable application of the technology is related to animal feed manufacturing processes and uses. However, the present composition is not limited to feed applications and may generally be used in applications where protection of compounds from the effects of high heat (e.g., >70° C.), pressure, oxidation, or water solubility is desired.

The present coating composition may be formed by combining a lipid with one or more hydrophobic, high melting point compounds, such as a fatty acid mineral salt, to form a coating with a significantly higher melting point. For example, commercially available forms of zinc stearate (“commercial grade zinc stearate”), which consist primarily of zinc salts of a mixture of varying amounts of stearic acid and palmitic acid, have a melting point of about 122° C. When zinc salts of this type are combined with commercially available stearic acid (“commercial grade stearic acid”), a greater than linear increase in melting point of the coating compound can be achieved. For example, commercial grade stearic acid is available primarily as a mixture of varying amounts of octadecanoic acid and hexadecanoic acid and typically has a melting point about 68° C. A combination of 50 wt. % commercial grade zinc stearate and 50 wt. % commercial grade stearic acid can have a melting point of about 105° C.

As employed herein, the terms “stearic acid” and “zinc stearate” refer to the chemically pure forms of these substances. Commercially available forms of these substances are expressly referred to herein as such and generally include substantial amounts of impurities. For example, as noted above, commercial grade stearic acid generally includes a substantial amount of palmitic acid. Commercial grade zinc stearate typically includes a mixture of zinc salts of stearic and palmitic acids together with a minor amount of zinc oxide. Also, as employed herein, the term “feed ingredients” refers to ingredients which may be included in an edible composition for consumption by animals and/or humans.

The present coating composition can be used with microencapsulation techniques to provide coated ingredients capable of better withstanding the effects of high heat (e.g., >70° C.), pressure, oxidation, and/or water solubility. One embodiment of the coating composition combines a lipid with one or more hydrophobic, high melting point compounds, such as a mineral salt of a fatty acid (e.g., zinc, calcium or magnesium stearates) thereby greatly improving the protection of the coated ingredient(s) from heat, air oxidation, chemical reactivity and/or water interaction. It is desirable, but not required, for all of the components in the coating composition to be edible. One suitable process described herein uses a coating formula which includes zinc salts of fatty acids, such as stearic, palmitic and/or lauric acid, in combination with animal tallow, vegetable stearin and/or saturated fatty acid(s) for illustrative purposes.

In another embodiment, the coating composition includes a solid solution including a zinc organic acid salt component and a lipid component. The melting point of the solid solution is commonly about 70° C. to 180° C. and coating compositions of this type in which the solid solution has a melting point of at least about 100° C. are particularly desirable. Very commonly, the zinc organic acid salt component is the zinc salt(s) of organic acid material having an Iodine Value not greater than about 20. Iodine Value is a measure for characterizing the average number of double bonds present in an organic acid which includes molecules with unsaturated residues. The Iodine Value of a material such as a mixture of fatty acids or mixture of triacylglycerols is determined by the Wijs method (A.O.C.S. Cd 1-25). For example, unprocessed soybean oil typically has an Iodine Value of about 125 to 135 and a pour point of about 0° C. to −10° C. Hydrogenation of soybean oil to reduce its Iodine Value to about 90 increases the melting point of the material as evidenced by the increased in its pour point to 10 to 20° C. Further hydrogenation can produce a material which is a solid at room temperature and may have a melting point of about 70° C.

In another embodiment, an encapsulated ingredient is disclosed. The encapsulated ingredient includes an ingredient component, such as a nutrient material in particulate form, and a coating component. The coating component can include a solid solution including a zinc organic acid salt component and a lipid component. The coating component commonly substantially surrounds the ingredient component. For example, particles of the ingredient may be substantially surrounded by a relatively thin layer of the coating composition or the ingredient particles may be embedded in a matrix of the coating material.

In another embodiment, an animal feed is disclosed comprising an encapsulated feed ingredient and feedstuff. The encapsulated feed ingredient includes an ingredient component and a coating component where the coating component typically substantially surrounds the ingredient component. The coating component can include a solid solution which includes a zinc organic acid salt component and a lipid component.

When combining a hydrophobic, high melting point compound and a lipid, the resulting coating may provide significant protection for ingredients even under the process conditions associated with extrusion and pelleting. As used herein, the term “protection” refers to a decrease in physical loss and/or loss of functionality under conditions associated with manufacturing, storage and/or use. In some instances, the “protection” can result in a complete prevention of such losses. In contrast, in the previous practice of using a coating formed solely from lipid, the coating can liquefy and rupture resulting in the loss of ingredient content and/or functional value during processing.

### BRIEF DESCRIPTION OF THE FIGURES

FIG. 1 shows a graph of the percent loss of methionine versus leaching time for unprotected methionine and methionine coated according to Example 1.

FIG. 2 shows a graph of the percent loss of Vitamin C versus leaching time for three samples of Vitamin C coated with the present coating compositions and one sample of commercially available Vitamin C coated with ethyl cellulose.

### DETAILED DESCRIPTION

The lipid/mineral salt coatings described herein can provide greater protection of ingredient content and functionality due to their relatively higher melting point.

The combination of one or more, hydrophobic, high melting point compounds (e.g., mineral salts of fatty acids such as commercial grade zinc stearate) with one or more type of lipid, forms a coating compound that can protect the coated ingredient(s) content and functionality. These coatings can be formulated to meet the needs of high temperature and pressure processing conditions.

The hydrophobic, high melting point compounds typically have a melting point of at least about 70° C. and, more desirably, greater than 100° C. Zinc salts of fatty acids having a melting point between about 115° C. and 130° C. are suitable hydrophobic, high melting point compounds. For this study, commercial grade zinc stearate was selected as a representative hydrophobic, high melting point compound from a group including, but not limited to:

| | |
|---|---|
| Metal carbonates | Calcium, magnesium, zinc carbonate |
| Metal silicates | Sodium or potassium silicates |
| Metal alginates | Calcium, magnesium, zinc alginates |
| Metal stearates | Zinc, calcium, magnesium stearates |
| Waxes | C26 and higher, parrafin, cholesterol |
| Fatty alcohols | Cetyl (hexadecanoic) alcohol |
| Polysaccharides | Chitin |
| Phospholipids | Lecithin |
| Mono, di and triglycerides of | Tallow, hydrogenated fat |
| animal and vegetable origin | |
| Fat derivatives | Fatty acids, soaps, esters |
| Hydrophobic starches | Dry-Flo (trade name of National |
| | Starch & Chemical |
| Proteins | Zein (protein from corn) |

The lipid component typically has a melting point of at least about 0° C. and more suitably no less-than about 40° C. The lipid component may include vegetable oil, such as soybean oil. In other embodiments, the lipid component may be a triacylglycerol with a melting point of about 45-75° C. Commercial grade stearic acid was selected as a representative lipid from a group including but not limited to: stearic acid, hydrogenated animal fat, animal fat (e.g., animal tallow), vegetable oil, such as crude vegetable oil and/or hydrogenated vegetable oil (either partially or fully hydrogenated), lecithin, palmitic acid, animal oils, wax, fatty acid esters-C8 to C24, fatty acids-C8 to C24.

Encapsulation of ascorbic acid, methionine and xylanase were performed to demonstrate the ability of the present method(s) to provide protection to a wide variety of ingredients. Ingredients which may be encapsulated include those which have nutritional applications and/or functional applications, such as gases, water, organic acids, and preservatives. Typically, the ingredient will be appropriate for use in feed and/or food. Suitable feed ingredients (i.e., ingredients for use in feed and/or food) include nutrients as well as other additives such as preservatives and medications (e.g., antibiotics). The ingredients may be selected from a group including but not limited to:

| | |
|---|---|
| Vitamins | C, B1, B6, B12, niacin, panthothenic acid, |
| | riboflavin, folic acid, biotin, choline, etc. |
| Fat soluble vitamins | A, D, E, K |
| Minerals | Calcium, iodine, iron, magnesium, manganese, |
| | phosphorus, selenium, sodium, zinc, cobalt, etc. |
| Nucleic acids/ | Guanine, adenine, thymidine, cytosine, uracil, etc. |
| nucleotides/other | |
| Amino acids | Valine, isoleucine, lysine, threonine, cystine, |
| | phenylalanine, alanine, methionine, histidine, |
| | leucine, arginine, glutamine, etc. |
| Enzymes | Xylanase, phytase, cellulase, peptidase, lipase, |
| | esterase, protease, glucanase, mannanase, amylase, |
| | chitinase etc. |
| Bacteria/fungi | A. oryzae, B. subtilis, B. licheniformis |
| Fatty acids | Omega-3 (alpha-linolenic, eicosapentaenoic acid, |
| | docosahexaenoic acid), omega-6 (linoleic, gamma |
| | linolenic, arachidonic), conjugated linoleic acid, etc. |
| Proteins | Isolated soy protein, soluble peptides and proteins, |
| | etc. |
| Pigments | Carotenoids, astaxanthin, zeaxanthin, |
| | canthaxanthin, beta-carotene, etc. |
| Medications and | Terramycin, erythromycin, oxytetracycline, |
| vaccines | sulfadimethoxine/ormetoprim, etc. |
| Sugars | Dextrose, sucrose, fructose, etc. |

The encapsulated ingredients may be prepared where the coating is present in an amount as low as 1% of the weight relative to the active ingredient and as high as 20 times the weight of the active ingredient giving it great flexibility. Commonly, the coating composition represents about 25 to 85 wt. % of the total weight of the coated ingrdient.

Leaching occurs when an unprotected ingredient is placed in a surrounding environment to which it is soluable resulting in loss of some of the ingredient itself to the environment or loss of some of its intended functionality within the animal. Leaching generally occurs in an aqueous or other liquid environment where the ingredient becomes exposed to the environment prior to reaching the intended site of use.

Previous practices of using lipid only coatings, were somewhat effective for leaching protection; However, according to the MERCK Index and many specification sheets on lipids, stearic acid and other types of lipids, are not completely insoluble in water. For example, stearic acid is slightly soluble in water so its use alone as a coating, results in inadequate leaching protection.

The present coatings which use one or more, hydrophobic, high melting point compounds combined with a lipid are more effective in protecting ingredients from the effects of leaching. For example, commercial grade zinc stearate is extremely hydrophobic and completely insoluble in water. When combined with a somewhat insoluble lipid such as commercial grade stearic acid, the coating compound is a better choice for prevention of ingredient loss in a watery medium.

The addition of commercial grade zinc stearate to the coating formula has improved the protection level of the ingredient and its functionality, significantly over a lipid only coating.

This benefit of the present coating composition can be utilized in feeds designed for ruminants. Because the ruminant's digestive track is in many ways similar to an aqueous or watery environment, it presents some of the same concerns related to leaching.

The present composition can be prepared in a number of ways. Preferably, the preparation process includes making a solid solution of the zinc organic salt component and the lipid component. In one embodiment, the solid solution can be formed by melting the lipid component and the zinc organic salt component until they both dissolve and allowing the solution to solidify. In addition to the zinc organic acid component and the lipid component, the coating composition may include other components that may or may not dissolve in the process of forming the solid solution. For example, the coating composition may include small amounts of zinc oxide and other elements or compounds.

After preparing the coating composition, it can then be used to prepare the protected ingredient. One suitable procedure for preparing the protected ingredient uses encapsulation technology, preferably microencapsulation technology. Microencapsulation is a process by which tiny amounts of a gas, liquid, or solid ingredient are enclosed or surrounded by a second material, in this case a coating composition, to shield the ingredient from the surrounding environment.

A number of microencapsulation processes could be used to prepare the protected ingredient such as spinning disk, spraying, co-extrusion, and other chemical methods such as complex coacervation, phase separation, and gelation. One suitable method of microencapsulation is the spinning disk method. The spinning disk method typically uses an emulsion or suspension including the ingredient and the coating composition. The emulsion or suspension is fed to the disk surface where it can form a thin wetted layer that, as the disk rotates, breaks up into airborne droplets from surface tension forces that induce thermodynamic instabilities. The resulting encapsulated ingredients may be individually coated in a generally spherical shape or embedded in a matrix of the coating composition. Because the emulsion or suspension is not extruded through orifices, this technique permits use of a higher viscosity coating and allows higher loading of the ingredient in the coating.

Data from testing on the effectiveness of our specifically formulated coating using commercial grade zinc stearate is presented with comparisons in Examples 1-7.

The addition of one or more hydrophobic, high melting point compounds, such as a mineral salt of a fatty acid, to lipids in various levels can improve the protective properties of an ingredient coating. Coatings may include lipids such as animal and vegetable tallows, waxes, and fatty acids including lower melting point fatty acids such as polyunsaturated fats (see, e.g. in previous lipid selection list). Hydrophobic, high melting point compounds (such as commercial grade zinc stearate) can be used in combination with lipids to increase their effectiveness in different applications and environments. The addition of an emulsifying agent such as glycerin, polysaccharides, lecithin, gelling agents and soaps, can improve the speed and effectiveness of the encapsulation process. Additionally, an anti-oxidant may be added to the coating formulation to provide improved protection against oxidation effects.

We have outlined the use of this invention for the protection of ingredients against heat and pressure experienced during the manufacturing process (pelleting and extrusion). Ingredients which most benefit from this form of protection are ingredients that are subject to heat damage or degradation including vitamins, pigments, proteins, amino acids, attractants and enzymes. This coating compound has application in all types of production processes where heat is applied and heat susceptible ingredients are used.

In addition to protecting ingredients from heat related damage or loss there is also the need to protect ingredients to damage or loss attributable to association or chemical reaction with other ingredients. For example this method of encapsulation provides the ability to prepackage a bundle of ingredients or combine usually exclusive ingredients in a formulation, while preventing harmful association with other ingredients.

For aquaculture and other liquid environment applications, this method of encapsulation results in a barrier to reduce leaching of water-soluble ingredients when they are introduced into the intended environment.

In some cases there may be a need to incorporate a gas into a feed or to entrap gas in a coating that contains other ingredients. Examples of where this is useful are to enhance the floating characteristics of a feed or to produce a floating feed with formulations previously known as challenging, or impossible to produce in floating form (such as a high fat and/or protein formulas).

A further application of the present composition is the ability to time or target release of a specific ingredient at a specific time or point in the digestive track. This may be a particularly desirable application under the unique conditions present in the rumen. The coating composition's ability to time or target release a specific ingredient at a certain point in the ruminent's digestive track is highly beneficial to the animal and improves cost effectiveness of ruminant formulations.

### ILLUSTRATIVE EMBODIMENTS

A number of illustrative embodiments of the present coating composition and its uses are described below. The embodiments described are intended to provide illustrative examples of the present coating composition and its uses and are not intended to limit the scope of the invention.

In one embodiment, the coating composition includes a solid solution including a zinc organic acid salt component and a lipid component. The zinc organic acid salt component commonly has an Iodine Value not greater than about 20 and in some instances the Iodine Value is no greater than about 10. The melting point of the solid solution is desirably at least about 70° C. Commonly, the solid solution has a melting point of at least about 90° C. and, more desirably, about 100° C. to 130° C. In order to avoid decomposition of the ingredient to be encapsulated, the melting point of the solid solution is generally no more than about 180° C. Suitably, the melting point of the lipid component is at least about 40° C., desirably at least about 45° C. and, commonly, no more than about 75° C. The lipid component may include animal tallow, stearic acid, hydrogenated vegetable oil, and/or vegetable stearin. The melting point of the zinc organic acid salt component is preferably at least about 100° C. and, more suitably, about 110° C. to 150° C. The zinc organic acid salt component can include at least about 80 wt. % zinc salt(s) of fatty acid material. The fatty acid material desirably has an Iodine Value not greater than about 10. For example, the zinc organic acid salt component can include at least about 80 wt. % zinc salt(s) of stearic acid, palmitic acid or a mixture thereof.

In another embodiment, the coating composition includes a lipid component and a solid solution, which includes a zinc organic acid salt component including at least about 80 wt. % zinc salts of saturated fatty acids. The saturated fatty acids typically have 14 to 22 carbon atoms. The melting point of the zinc organic acid salt component is commonly at least about 90° C. and, more desirably, about 100° C. to 130° C. For example, the zinc organic acid salt component can include a mixture of zinc salts of stearic and palmitic acid and have a melting point of about 115° C. to 130° C. The lipid component may include animal tallow, stearic acid, hydrogenated vegetable oil, and/or vegetable stearin. Suitably, the melting point of the lipid component is at least about 40° C., desirably at least about 45° C. and, commonly, no more than about 75° C. The melting point of the solid solution is desirably at least about 70° C. Commonly, the solid solution has a melting point of at least about 90° C. and, more desirably, about 100° C. to 130° C.

In yet another embodiment, the coating composition includes a lipid component and a solid solution including zinc salts of fatty acid material. The fatty acid material commonly has an Iodine Value of no more than about 20. The coating composition often includes at least about 40 wt. % of the zinc salts of the fatty acid material. The melting point of the solid solution is generally is about 90° C. to 130° C. and, more suitably at least about 100° C. Typically, the melting point of the lipid component is at least about 40° C. and, more commonly about 45° C. to 75° C. The lipid component can include animal tallow, stearic acid, hydrogenated vegetable oil, vegetable stearin, or mixtures thereof. The melting point of the zinc salts is suitably about 100° C. to 180° C. and more preferably no more than about 150° C. Quite commonly, the melting point of the zinc salts is about 115° C. to 130° C. For example, the zinc salt can have an Iodine Value not greater than about 10 and includes at least about 80 wt. % zinc salt(s) of stearic acid, palmitic acid or a mixture thereof.

In another embodiment, an encapsulated ingredient, which includes an ingredient component and a coating component, is disclosed. The ingredient component is typically substantially surrounded by the coating component. The coated ingredient may be in a particulate form, which includes the ingredient component substantially surrounded by a layer of the coating component. In other instances, the coated ingredient may be in a particulate form, which includes the ingredient component embedded in a matrix of the coating component. The ingredient component may include one or more nutrients or other feed ingredients. For example, the ingredient component may include a nutrient such as ascorbic acid, an amino acid (e.g., methionine), or a protein source (e.g., soy protein isolate). Typically, the encapsulated ingredient includes about 25 to 95 wt. % of the coating component, suitably about 40 to 85 wt. %, and more commonly about 50 to 75 wt. %. The encapsulated ingredient generally includes at least about 10 wt. % of the ingredient component, more commonly about 15 to 45 wt. %, and suitably 20 to 40 wt. %. The coating component may include a solid solution including a zinc organic acid salt component and a lipid component. Suitably, the coating component includes at least about 10 wt. % zinc salt(s) of fatty acid material, which typically has an Iodine Value not greater than about 10. The melting point of the zinc fatty acid salt material is suitably about 90° C. to 150° C. and, preferably, about 100° C. to 130° C. Commonly, the solid solution has a melting point of at least about 90° C. and, more desirably, about 100° C. to 130° C. In order to avoid decomposition of the ingredient to be encapsulated, the melting point of the solid solution is generally no more than about 180° C.

In another embodiment, an encapsulated ingredient, which includes an ingredient component and a coating component substantially surrounding the ingredient component, is disclosed. The coating component includes a solid solution including a zinc organic acid salt component and a lipid component. The melting point of the solid solution is desirably at least about 70° C. Commonly, the solid solution has a melting point of at least about 90° C. and, more desirably, about 100° C. to 130° C. The zinc organic acid salt component commonly has an Iodine Value not greater than about 20 and in some instances the Iodine Value is no greater than about 10. Suitably, the melting point of the lipid component is at least about 40° C., desirably at least about 45° C. and, commonly, no more than about 75° C. The lipid component may include animal tallow,. stearic acid, hydrogenated vegetable oil, and/or vegetable stearin. The melting point of the zinc organic acid salt component is preferably at least about 100° C. and, more suitably, about 110° C. to 150° C. The zinc organic acid salt component can include at least about 80 wt. % zinc salt(s) of fatty acid material. The fatty acid material desirably has an Iodine Value not greater than about 10. For example, the zinc organic acid salt component can include at least about 80 wt. % zinc salt(s) of stearic acid, palmitic acid or a mixture thereof.

Another embodiment discloses an animal feed which includes the encapsulated ingredient and feedstuff. The encapsulated ingredient includes an ingredient component and a coating component. The ingredient component may include a nutrient such as a protein source, a vitamin, an enzyme, an amino acid, and/or an sugar. The coating component includes a solid solution, which includes a zinc organic acid salt component and a lipid component. Commonly, the solid solution has a melting point of at least about 90° C. and, more desirably, about 100° C. to 130° C. The zinc organic acid salt component may include zinc salt(s) of fatty acid material, e.g., zinc salt(s) of stearic and/or palmitic acid having a melting point of 115° C. to 130° C. The lipid component commonly includes materials such as animal tallow, stearic acid, hydrogenated vegetable oil, and/or vegetable stearin. Suitably, the melting point of the lipid component is at least about 40° C., desirably at least about 45° C. and, commonly, no more than about 75° C.

In another embodiment, an encapsulated ingredient, which includes an ingredient component and a coating component, is disclosed. The coating component includes a lipid and one or more hydrophobic, high melting point compounds. The coating component has a melting point of at least about 70° C. and, more suitably, at least about 100° C. The ingredient component is typically substantially surrounded by the coating component. The coated ingredient may be in a particulate form, which includes the ingredient component substantially surrounded by a layer of the coating component. In other instances, the coated ingredient may be in a particulate form, which includes the ingredient component embedded in a matrix of the coating component.

Another embodiment discloses an animal feed which includes the encapsulated ingredient and feedstuff. The encapsulated ingredient is typically in particulate form and includes an ingredient component and a coating component. The coating component includes a lipid and one or more hydrophobic, high melting point compounds. The coating component has a melting point of at least about 70° C. and, more suitably, at least about 100° C. The ingredient component may include a nutrient such as a protein source, a vitamin, an enzyme, an amino acid, a nucleic acid, a mineral, a fatty acid, and/or a sugar.

In another embodiment, a coating component is disclosed. The coating component includes a lipid and one or more hydrophobic, high melting point compounds. The coating component also has a melting point of at least about 70° C. and, more suitably, at least about 100° C.

### EXAMPLES

The following examples are presented to illustrate the present invention and to assist one of ordinary skill in making and using the same. The examples are not intended in any way to otherwise limit the scope of the invention.

### Example 1

Coated methionine was formed using the following procedure. Initially, the coating composition was prepared by forming a liquid solution of 50 wt. % commercial grade zinc stearate, available from Acme-Hardesty, Inc., Blue Bell, Pa., and 50 wt. % commercial grade stearic acid. The liquid solution was formed by feeding commercial grade zinc stearate and commercial grade stearic acid into a holding tank and heating the mixture to just above its melting point, in this case about 120° C. The resulting molten material included a homogeneous liquid phase containing fatty acids and zinc fatty acid salts.

It should be noted that commercial grade zinc stearate used in this and the following examples is commercially available in large quantities and as such is not pure zinc distearate. Rather, commercial grade zinc stearate is made up primarily of variable proportions of zinc salts of stearic and palmitic acid together with small amounts of other elements and compounds such as zinc oxide. Also, the commercial grade stearic acid used in this example is commercially available in large quantities and is not pure octadecanoic-acid. Rather, commercial grade stearic acid is available primarily as a mixture of variable proportions of octadecanoic acid (“stearic acid”) and hexadecanoic acid (“palmitic acid”) along with various amounts of other fatty acids.

The methionine was prepared by passing it through a screen to ensure that the particle size was smaller than 100 microns. After screening the methionine, it was fed to a slurry vessel where it was combined with the coating composition. In this example, the methionine and coating composition were delivered to the slurry vessel at the rate of 100 lb/hr to form a 50/50 wt. % slurry.

The molten coating composition and methionine were mixed in the slurry vessel for no more than 10 seconds. The mixing time was minimized to prevent the methionine from being damaged. Upon exiting the slurry vessel, the slurry was gravity fed to the surface of a heated rotating disk rotating at 750 rpm. As the disk rotated, the slurry spread across it due to centrifugal forces. At the edge of the disk the slurry was sheared into particles that allowed the coating to surround the methionine. As the particles of coated methionine fell from the disk to a collection hopper, the coating composition cooled and solidified.

### Example 2

A nutrient, ascorbic acid, coated according to the method described in Example 1 was compared to ascorbic acid coated only with stearic acid to determine which coating was most effective at protecting the ascorbic acid during extrusion processing. The ascorbic acid coated according to the method described in Example 1 included about 65 wt. % of the coating component, a 50/50 wt. % blend of zinc stearate and stearic acid, with the remainder (35 wt. %) being ascorbic acid. The other sample of coated ascorbic acid included about 65% of stearic acid with the remainder being ascorbic acid. Equal levels of each sample (circa 0.02 wt. % based on total feed weight) were introduced into a feed formula that was then processed by extrusion. The level of ascorbic acid in the end product was tested and resulted in the following:

| | LOSS OF |
|---|---|
| COATING | VITAMIN C |
| Ascorbic acid coated with commercial grade stearic acid | 60% |
| Ascorbic acid coated with 50/50 wt. % commercial grade | 20% |
| stearic acid-commercial grade zinc stearate | |

### Example 3

Methionine coated according to the method described in Example 1 was compared to uncoated methionine to determine which coating was most effective at preventing leaching of the methionine in an aqueous environment. Methionine coated according to the method described in Example 1 included about 75 wt. % of the coating component, a 50/50 wt. % blend of commercial grades of zinc stearate and stearic acid, with the remainder being methionine. Equal levels of each sample (circa 1.0 wt. % based on total feed weight) were introduced into a feed formula that was placed in a vessel and contacted with deionized water. The level of methionine in the end product was tested and resulted in the following:

| | LEACHING LOSS AFTER |
|---|---|
| PROTECTED METHIONINE FORM | 30 MINUTES |
| Uncoated methionine | 40% |
| Methionine coated with 50/50 wt. % | 13% |
| commercial grade stearic acid- | |
| commercial grade zinc stearate | |

### Example 4

A sample of protein was coated according to the method described in Example 1. The coated protein was compared to a sample of uncoated protein to test the coating's ability to time, or target release, a specific ingredient within the rumen through slowing leaching effects. The protein coated according to the method described in Example 1 included about 50 wt. % of the coating component, a 50/50 wt. % blend of commercial grade zinc stearate and animal tallow, with the remainder (50 wt. %) being protein. Equal levels of each sample (circa 10-15 grams) were placed in a fermentation vessel and contacted with rumen fluid. Protein losses were reduced by 50% versus that of uncoated protein as demonstrated below.

| | PROTEIN LOSS IN RUMEN |
|---|---|
| COATING | (24 hrs.) |
| Uncoated protein | 40% |
| Protein coated with 50/50 wt. % | 20% |
| commercial grade zinc stearate - animal | |
| tallow | |

### Example 5

A coated material containing an enzyme, such as xylanase having a specific activity of 27,000 IU/gm, was formed using the following procedure. The coating composition can be prepared by forming a liquid solution of 10 wt. % commercial grade zinc stearate and 90 wt. % commercial grade stearic acid. If desired animal tallow and/or vegetable stearine can be used in place of part or all of the commercial grade stearic acid. This was done by feeding commercial grades of zinc stearate and stearic acid into a holding tank and heating the mixture to just above its melting point, in this case 120° C.

After preparing the coating composition, the enzyme material and coating were prepared by emulsifying the enzyme in the molten coating composition together with 0.5 wt. % of an emulsifying agent, such as lecithin, in a slurry vessel.

The molten coating composition and material were mixed in the slurry vessel, typically for no more than 10 seconds. The mixing time was minimized to prevent the enzyme material from being damaged. Upon exiting the slurry vessel, the slurry was gravity fed to the surface of a heated rotating disk rotating at 750 rpm. As the disk rotates, the emulsion spreads across it due to centrifugal forces. At the edge of the disk the slurry was sheared into discrete droplets allowing the coating to surround the material. As the coated material falls from the disk to a collection hopper, the coating composition cools and solidifies.

The material coated according to the above described method can be compared to the same material that has not been coated and is in a free form. Xylanase enzyme in an unprotected form will commonly lose 80 wt. % or more of activity when subjected to the heat conditions of pelleting (90° C. for periods over 1 minute) as part of an animal feed. A mixture of 80% coating (10 wt. % commercial grade zinc stearate and 90 wt. % commercial grade stearic acid) and 20 wt. % xylanase enzyme was prepared by encapsulating with the spin disk method and subjected to 90° C. heat for 5 minutes. The results are as follows:

| | MATERIAL ACTIVITY LOSS |
|---|---|
| PROTECTED MATERIAL FORM | AFTER 5 MINUTES |
| Material coated with 90/10 wt. % | 3% |
| commercial grade stearic acid- | |
| commercial grade zinc stearate | |

### Example 6

Methionine coated according to the method described in Example 1 was compared to unprotected methionine to determine the degree to which the coating prevented leaching of the methionine in an aqueous environment. Methionine coated according to the method described in Example 1 included about 75 wt. % of the coating component, a 50/50 wt. % blend of commercial grades of zinc stearate and stearic acid, with the remainder (25 wt. %) being methionine. Equal levels of each sample (circa 1.0 wt. % of methionine based on total feed weight) were introduced into a feed formula that was placed in a vessel and exposed to deionized water for an hour.

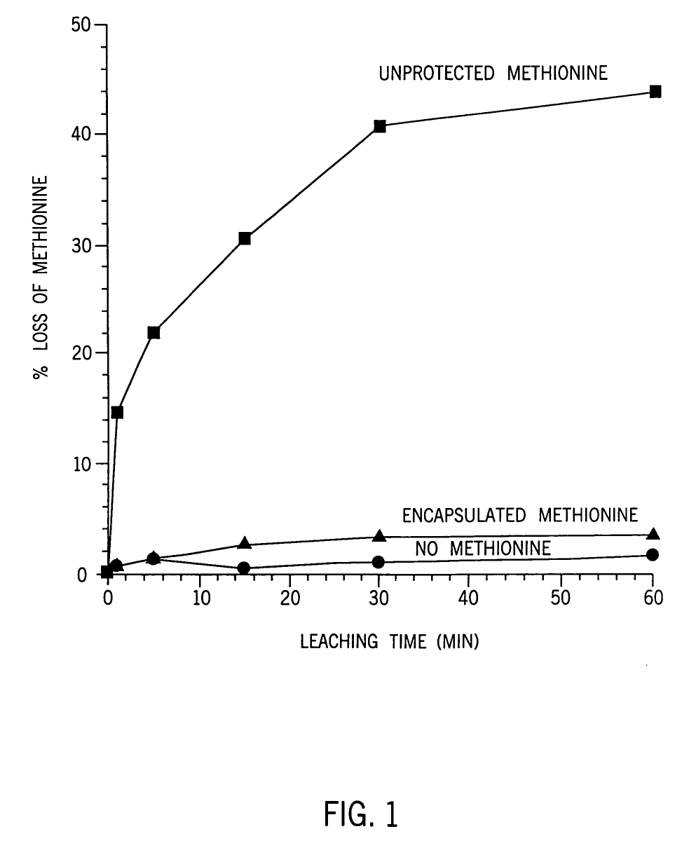

FIG. 1 is a graph of the results of the tests and shows the percent loss of methionine as a function of leaching time. As shown in FIG. 1, the percent loss of unprotected methionine increased quickly and then leveled off. Specifically, the unprotected methionine lost about 15 wt. % after about 1 minute, about 22 wt. % after about 5 minutes, about 30 wt. % after about 15 minutes, and about 43 wt. % after about 60 minutes. In contrast, the percent loss of the encapsulated methionine increased slowly and leveled off relatively quickly. Specifically, the encapsulated methionine lost about 1 wt. % after about 5 minutes, about 2 wt. % after about 15 minutes, and leveled off at about 2.5 wt. % loss after about 20 to 30 minutes.

### Example 7

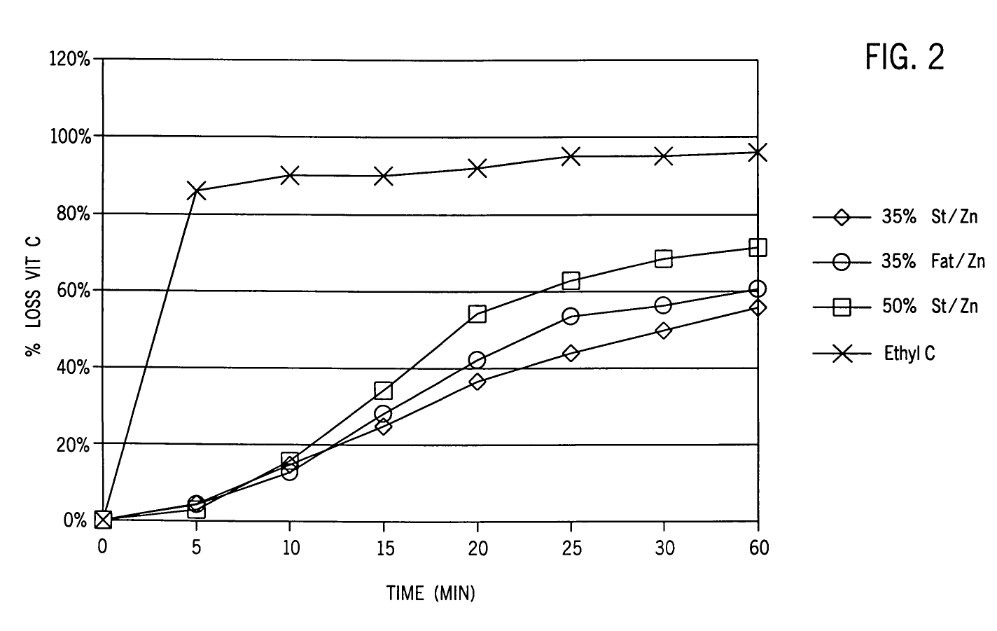

Leaching tests were performed on four samples of protected ascorbic acid to determine each sample's ability to prevent leaching of the ascorbic acid in a watery medium. Three of the samples were coated according to the method described in Example 1 with various combinations and amounts of lipids and commercial grade zinc stearate. The first sample of coated ascorbic acid (referred to as “35% St/Zn” in FIG. 2) included about 65 wt. % of the coating component, a 50/50 wt. % blend of commercial grades of zinc stearate and stearic acid, with the remainder (35 wt. %) being ascorbic acid. The second sample of coated ascorbic acid (referred to as “35 wt. % Fat/Zn” in FIG. 2) included about 65 wt. % of the coating component, a 50/50 wt. % blend of commercial grade zinc stearate and animal tallow, with the remainder (35 wt. %) being ascorbic acid. The third sample of coated ascorbic acid (referred to as “50 wt. % St/Zn” in FIG. 2) included about 50 wt. % of the coating component, a 50/50 wt. % blend of commercial grades of zinc stearate and stearic acid, with the remainder (50 wt. %) being ascorbic acid. The fourth sample was coated with ethyl cellulose (“Ethyl C”) and is generally available as a commercial product.

The leaching tests were performed by introducing equal levels of each sample (circa 1.0 wt. % based on total feed weight) into a feed formula that was placed in a vessel and exposed to deionized water. The level of ascorbic acid at various times was measured and the results are shown in FIG. 2 as a graph of percent loss of Vitamin C versus time. As shown in FIG. 2, the percent loss of Ethyl C was about 86 wt. % after about 5 minutes. Thereafter, the loss of Ethyl C leveled off so that after about 60 minutes about 97 wt. % was lost. In contrast, the ascorbic acid coated according to the method described in Example 1 lost ascorbic acid at a slower rate and after about 60 minutes lost less ascorbic acid overall than the Ethyl C.

The invention has been described with reference to various specific and illustrative embodiments, examples and techniques. However, it should be understood that many variations and modifications may be made while remaining within the spirit and scope of the invention.

What is claimed is:

1. An animal feed comprising:
feedstuff; and
an encapsulated ingredient including
an ingredient component; and
a coating component comprising a solid solution which includes a zinc organic acid salt component and a lipid component;
wherein the coating component includes no more than about 50 wt. % of the zinc organic acid salt component; and
wherein the coating component substantially surrounds the ingredient component.

2. The animal feed of claim 1 wherein the lipid component includes a material selected from the group consisting of animal fat, fatty acid, vegetable oil, and mixtures thereof.

3. The animal feed of claim 1 wherein the ingredient component includes ascorbic acid.

4. The animal feed of claim 1 wherein the ingredient component includes at least one of an amino acid or an enzyme.

5. The animal feed of claim 1 wherein the solid solution has a melting point of about 90° C. to 130° C.

6. The animal feed of claim 1 wherein the zinc organic acid salt component includes at least about 80 wt. % of zinc salts of at least one of stearic acid or palmitic acid.

7. The animal feed of claim 1 comprising:
about 50 to 75 wt. % of the coating component; and
about 25 to 50 wt. % of the ingredient component.

8. The animal feed of claim 1 wherein the coating component comprises at least about 10 wt. % of the zinc organic acid salt component.

9. The animal feed of claim 1 wherein the encapsulated ingredient includes no more than about 45 wt. % of the ingredient component.

10. An animal feed comprising:
feedstuff; and
an encapsulated ingredient including
no more than about 45 wt. % ingredient component; and
a coating component comprising a solid solution which includes a zinc fatty acid salt component and a lipid component;
wherein the ingredient component is substantially surrounded by the coating component.

11. The animal feed of claim 10 wherein the ingredient component includes ascorbic acid.

12. The animal feed of claim 10 wherein the ingredient component includes at least one of an amino acid or an enzyme.

13. The animal feed of claim 10 wherein the zinc fatty acid salt component has a melting point of about 100° C. to 180° C.

14. The animal feed of claim 10 wherein the solid solution has a melting point of about 90° C. to 130° C.

15. The animal feed of claim 10 wherein the coating component comprises at least about 10 wt. % of the zinc organic acid salt component.

16. An animal feed comprising:
feedstuff; and
an encapsulated ingredient including
an ingredient component; and
a coating component comprising a solid solution which includes a zinc organic acid salt component and a lipid component;
wherein the solid solution has a melting point of about 90° C. to 130° C.; and
wherein the ingredient component is substantially surrounded by the coating component.

17. The animal feed of claim 16 wherein the lipid component includes a lipid selected from the group consisting of animal fat, stearic acid, palmitic acid, vegetable oil, and mixtures thereof.

18. The animal feed of claim 16 wherein the ingredient component includes ascorbic acid.

19. The animal feed of claim 16 wherein the ingredient component includes at least one of an amino acid or an enzyme.

20. The animal feed of claim 16 wherein the zinc organic acid salt component includes at least about 80 wt. % of zinc salts of at least one of stearic acid or palmitic acid.

21. The animal feed of claim 16 comprising:
about 50 to 75 wt. % of the coating component; and
about 25 to 50 wt. % of the ingredient component.

22. The animal feed of claim 16 wherein the coating component comprises no more than about 10 wt. % of the zinc organic acid salt component.

23. The animal feed of claim 16 wherein the encapsulated ingredient includes no more than about 45 wt. % of the ingredient component.
